(19) United States
(12) Patent Application Publication
(10) Pub. No.: US 2013/0091611 A1
(43) Pub. Date: Apr. 18, 2013

(54) Anti-viral Formulations Nanomaterials and Nanoparticles

(71) Applicants: Guogang Ren, London (GB); John S. Oxford, Prof., London (GB); Paul William Reip, Farnborough (GB); Robert Lambkin-Williams, Brighton (GB); Alexander Mann, London (GB)

(72) Inventors: Guogang Ren, London (GB); John S. Oxford, Prof., London (GB); Paul William Reip, Farnborough (GB); Robert Lambkin-Williams, Brighton (GB); Alexander Mann, London (GB)

(21) Appl. No.: 13/691,099

(22) Filed: Nov. 30, 2012

Related U.S. Application Data
(63) Continuation of application No. 12/279,627, filed on Jul. 27, 2009, now abandoned.

(30) Foreign Application Priority Data
Feb. 16, 2006 (GB) 0603138.9

(51) Int. Cl. A41D 13/11; A01N 25/34

(52) U.S. Cl. CPC A41D 13/1192; A01N 25/34; B82Y 5/00
USPC 2/9; 424/601; 424/618; 424/630; 424/635; 424/641; 424/646; 424/724; 424/600; 514/63; 514/492; 442/330; 96/226; 2/455; 977/904

(57) ABSTRACT

The present invention provides the use of nanoparticles of a compound of general formula MₙX_{y}, where M is (i) a metal selected from the group consisting of Calcium (Ca), Aluminium (Al), Zinc (Zn), Nickel (Ni), Tungsten (W) or Copper (Cu); or (ii) a non-metal selected from the group consisting of Silicon (Si), Boron (B) or Carbon (C); in which n is equal to 1, 2, or 3, and X is (iii) a non-metal selected from the group consisting of Oxygen (O), Nitrogen (N), or Carbon (C); or (iv) an anion selected from the group consisting of phosphate (PO₄³⁻), hydrogen phosphate (HPO₄²⁻), dihydrogen phosphate (H₂PO₄⁻), carbonate (CO₃), silicate (SiO₄²⁻), sulphate (SO₄²⁻), nitrate (NO₃⁻), nitrite (NO₂⁻); in which y is equal to 0, 1, 2, 3 or 4; for use in reducing and/or preventing virus transmission. Articles of protective clothing or filters are provided in which the fibres are coated with said nanoparticles for use in reducing and/or preventing virus transmission.

The present invention relates to the use of nanoparticles of metals and/or metal compounds in the prevention of viral infection.

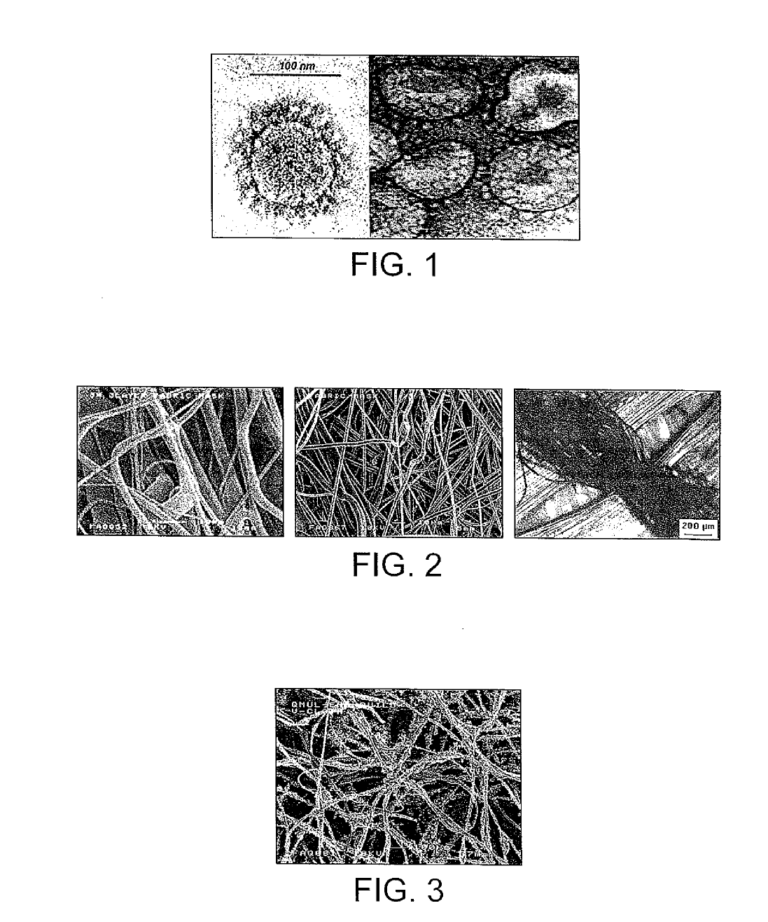

Airborne viral infection is commonly caused by inhalation of droplets of moisture containing virus particles. Larger virus-containing droplets are deposited in the nose, while smaller droplets or nano particles find their way into the human airways or alveoli. The SARS virus as shown in FIG. 1 is spread by droplets produced by coughing and sneezing with the sizes around 100-500 nm although other routes of infection may also be involved, such as facial contamination (Donnelly et al. Lancet, 361, 1761-1777, (2003)). From a filtration point of view, nano-scaled viruses and particles can therefore theoretically penetrate through the gaps of normal facial marks. The diameter of current world superfine artificial or natural fibre filaments is around 7 micrometers. The standard facial mask as shown in FIG. 2 has around >20-10 μm gaps all the way around the fibre mats.

Facial masks using traditional filtration fabric materials are therefore inadequate for stopping nano-scaled viruses. The gaps among fibers on facial mask are on average 10 to 30 μm (10,000-30,000 nm). Masks with smaller fibre gaps will result in breathing difficulty. Other nano-scaled airborne viruses and particles as smoke and super fine dust can enter into human lungs and then into blood system through respiratory membranes. The health effect is related mainly to the sub-micron sized fraction of the particles (i.e. an aerodynamic diameter, dₚ, less than 1 μm). The danger from smoke particles is the dₚ<100 nm fraction and such small particles are generated in huge amounts in the combustion processes.

Particles smaller than 100 nm are nanomaterials covering a range of sizes including that of human viruses such as Avian influenza and HIV. The global concern about Infuenza (i.e. the result of SARS and H5N1 viral infection) and AIDS are now well identified problems in the modern world but solutions to help prevent the spread of viral disease haven been lacking so far. However, nanomaterials may provide vital solutions for humans to conquer these diseases. Solutions are urgently required to deal with these epidemics.

Nanoparticles can be characterized by electron microscopy, for example transmission or scanning electron microscopy (TEM or SEM), atomic force microscopy (AFM), x-ray photoelectron spectroscopy (XPS), powder x-ray diffractometry (XRD), and Fourier transform infrared spectroscopy (FTIR).

Nanoparticles have found use in pharmaceutical formulations to improve solubility and/or biological activity of drug substances. In addition to pharmaceutical or research purposes, nanoparticles have been also been used for medical purposes. For example, silver nanoparticles have been used to kill bacteria (Furno et al J. Antimicrob Chemother, α(6), 1019-24 (2004)).

Other studies have described the use of nanometer catalysts which have been prepared with metals such as silver, titanium dioxide, zinc oxide and carbon (Fang et al Virologica Sinica, 20, 70-74 (2005). Such catalysts are supported nanometer-sized catalytic crystal particle compositions of metals wherein the exposed faces of the nanometer-sized catalyst particles comprise predominantly crystal planes of the (111) type. Such catalysts have been used to facilitate the dissociative adsorption, surface reaction, and recombination/desorption of hydrogen various hydrogenations and related reactions such as methanation, carbonylation, hydroformylation, reductive alkylation, amination, hydrosilation, ammonia synthesis, oil or fat hardening and the like. However, there has been no suggestion that a nanoparticle of metal or metal oxide could itself have any virucidal properties.

Other studies in the field of virology have investigated the use of materials such as Bentonite, which is a colloidal clay material. Nanoparticles of bentonite have been prepared and have been reported to have a virucidal activity. However, due to the complex nature of the material it is not clear whether the mechanism of action is tied to the particle size or to the inherent properties of the material (http://www.eswiconference.org-2005).

Recently, there have been reports (www.nanoscale.com) of the use of nanoparticulate silver being effective as an agent to prevent viral replication. However, the data does not suggest any virucidal effectiveness of the particles used over the physico-chemical characteristics of the material itself (Elechiguerra et al J. Nanobiotechnology 3 (6) (2005)). However, the use of silver is not 100% wholly effective and there are associated cost and toxicity problems.

The impact of virus outbreaks of SARS, avian flu and human influenza show how limited the current repertoire of defenses are against viral infection. Accordingly there is a need for improved means to prevent transmission of virus particles.

According to a first aspect of the invention, there is provided the use of nanoparticles of a compound of general formula MₙX_{y}, where M is
(i) a metal selected from the group consisting of Calcium (Ca), Aluminium (Al), Zinc (Zn), Nickel (Ni), Tungsten (W) or Copper (Cu);
or (ii) a non-metal selected from the group consisting of Silicon (Si), Boron (B) or Carbon (C);
   in which n is equal to 1, 2 or 3,

### and X is

(iii) a non-metal selected from the group consisting of Oxygen (O), Nitrogen (N), or Carbon (C);
or (iv) an anion selected from the group consisting of phosphate (PO₄³⁻), hydrogen phosphate (HPO₄²⁻), dihydrogen phosphate (H₂PO₄⁻), carbonate (CO₃), silicate (SiO₄²⁻), sulphate (SO₄²⁻), nitrate (NO₃⁻), nitrite (NO₂⁻);
   in which y is equal to 0, 1, 2, 3 or 4;
   for reducing and/or preventing virus transmission.

By nanoparticle is meant particles having nanometric dimensions, and nanoparticles may have, for example, dimensions in the order of a few nanometres to several hundred nanometres. The nanoparticles may be of a similar size to or smaller size than any given target virus or viruses.

Nanoparticles for use according to the present invention may have an average particle size of up to about 100 nm, up to about 200 nm, up to about 300 nm, or up to about 500 nm. Preferred average particle sizes may be in ranges of from about 1 nm to about 90 nm, suitably from about 5 nm to about 75 nm or from about 20 nm to about 50 nm. Particularly preferred average particle size ranges are of from about 20 nm to about 50 nm.

Preferred specific surface area of said particles may be in the range of from 150 m²/g to about 1450 m²/g, preferably, from 200 m²/g to about 700 m²/g, suitable values may comprise 150 m²/g, 640 m²/g, 700 m²/g. The voids in the particles may be of the order of from 0.1 to about 0.8 ml/g, suitably of from 0.2 to about 0.7 ml/g, preferably about 0.6 ml/g.

It is generally preferred that the nanoparticles are in the form of dry powders, but may also be in the form of liquids, sol-gels or polymers, as well as nanotubes. The particles may be agglomerated or in free association.

The nanoparticles may comprise single element M for the case where y is equal to 0 in the general formula Mₙ; and X is therefore not present, or the nanoparticles may comprise a compound as defined above where y has the value 1, 2 or 3 and x varies accordingly with respect to the value of y in conformity with the respective valencies of the elements M and X present in the formula.

Alternatively, the nanoparticles of a single element where y is equal to 0 may be doped with one or more elements selected from the group consisting of Silicon (Si), Boron (B), Phosphorous (P), Arsenic (As), Sulphur (S) or Gallium (Ga); alloyed with one or more elements selected from the group consisting of Aluminium (Al), Manganese (Mn), Magnesium (Mg), Nickel (Ni), Tin (Sn), copper (Cu), Titanium (Ti), Tungsten (W), Silver (Ag) or Iron (Fe).

For example, mixed nanoparticles may be composed of different elements as follows:

C—P—Ag—Zn, C—P—Cu—S, C—P—Cu—Ni—S, C—Si—Ag—Zn, C—Si—Cu—S, C—Si—Cu—Ni, C—Cu—Zn—W, C—Cu—Zn—Ag, C—Cu—Zn—W—Ag, C—W—Ti—B, C—W—Ti—N, C—Ti—N, Si—N, Ti—N, Al—N, B—N, Al—B.

The nanoparticles may also further comprise at least one of the following oxides: TiO₂, Cu₂O, CuO, ZnO, NiO, Al₂O₃, FeO, Fe₂O₃, Fe₃O₄, CoO, Co₃O₄, or Si₂O₃, or a combination thereof.

Preferred compounds of the general formula MₙX_{y} may be oxides, carbonates, silicates, carbides, nitrides and/or phosphates.

For example, aluminium oxide (Al₂O₃), silicon dioxide, (SiO₂), zinc oxide (ZnO), aluminium phosphate (i.e. aluminium phosphate (AlPO₄), aluminium hydrogen phosphate (Al₂(HPO₄)₃), aluminium dihydrogen phosphate (Al(H₂PO₄)₃), calcium oxide (CaO), calcium carbonate (CaCO₃), calcium silicate (CaSiO₄), calcium phosphate (i.e. calcium phosphate (Ca₃(PO₄)₂), calcium hydrogen phosphate (CaHPO₄), or calcium dihydrogen phosphate (Ca(H₂PO₄)), silicon nitride (Si₃N₄), silicon carbide (SiC), boron nitride (BN), tungsten carbide (WC), titanium carbide (TiC) or titanium carbonitride (TiC_{0.5}N_{0.5}).

The nanoparticles may also be prepared as layered (core/shell) particles comprising an inner core and an outer shell.

Other embodiments of the invention, may comprise the use of mixed compositions of nanoparticles. So, for example, a mixed composition may comprise one or more compounds of general formula MₙX_{y} as above (i.e. at least two such compounds), or may further comprise additional elements selected from the group consisting of: Boron (B), Carbon (C), Aluminium, (Al), Silicon (Si), Phosphorous (P), Calcium (Ca), Titanium (Ti), Chromium (Cr), Manganese (Mn), Iron (Fe), Cobalt (Co), Silver (Ag), Zinc (Zn), Copper (Cu), Sulfur (S), Nickel (Ni), Gold (Au), Zirconium (Zr), Ytterbium (Yb), Zirconium (Zr), or an oxide thereof or a combination thereof. Preferred oxides may include, for example titanium dioxide (TiO₂) or zirconium oxide (ZrO₂).

The mixed composition of nanoparticle may be Copper (Cu), copper (II) oxide (CuO) and/or copper (I) oxide (Cu₂O). The nanoparticles may comprise a mixed composition of a compound of general formula MₙX_{y} as defined in accordance with the first aspect and one or more of Aluminium (Al), Silicon (Si), Zinc (Zn), or Nickel (Ni), or combinations thereof. In such embodiments, the nanoparticles may comprise:
(i) Aluminium (Al) and aluminium oxide (Al₂O₃),
(ii) Silicon (Si) and silicon dioxide (SiO₂)
(iii) Silicon (Si) and Silicon carbide (SiC)
(iv) Zinc (Zn) and zinc oxide (ZnO), or
(v) Nickel (Ni) and nickel (II) oxide (NiO)
or combinations thereof.

The nanoparticles may further comprise one or more of titanium dioxide (TiO₂), zinc oxide (ZnO) and titanium dioxide (TiO₂).

Mixtures of nanoparticles of more than one of the above may also be prepared and used according to the present invention. Mixed nanomaterial compositions may be produced by any suitable method, such as for example, tumble-mixing, co-deposition, or mechanical alloying.

Uses in Accordance with the Present Invention Therefore Also Extend to Mixed-Oxide, Non-Stoichiometric Particles

Nanoparticle synthesis can be considered to comprise two main areas: gas phase synthesis and sol-gel processing. Nanoparticles may be generated by evaporation and condensation (nucleation and growth) in a subatmospheric inert-gas environment. Various aerosol processing techniques may be used to improve the production yield of nanoparticles. These include synthesis by combustion flame, plasma, laser ablation, chemical vapor condensation, spray pyrolysis, electrospray and plasma spray.

Sol-gel processing is a wet chemical synthesis approach that can be used to generate nanoparticles by gelation, precipitation, and hydrothermal treatment. Size distribution of semiconductor, metal, and metal oxide nanoparticles can be manipulated by either dopant introduction or heat treatment. Better size and stability control of quantum-confined semiconductor nanoparticles can be achieved through the use of inverted micelles, polymer matrix architecture based on block copolymers or polymer blends, porous glasses, and ex-situ particle-capping techniques.

Other nanoparticle synthesis techniques include sonochemical processing, cavitation processing (e.g. using a piston gap homogeniser), microemulsion processing, and high-energy ball milling. In sonochemistry, an acoustic cavitation process can generate a transient localized hot zone with extremely high temperature gradient and pressure. Such sudden changes in temperature and pressure assist the destruction of the sonochemical precursor (e.g., organometallic solution) and the formation of nanoparticles.

In hydrodynamic cavitation, nanoparticles are generated through creation and release of gas bubbles inside the sol-gel solution. By rapidly pressurizing in a supercritical drying chamber and exposing to cavitational disturbance and high temperature heating, the sol-gel solution is mixed. The erupted hydrodynamic bubbles are responsible for nucleation, growth, and quenching of the nanoparticles. Particle size can be controlled by adjusting the pressure and the solution retention time in the cavitation chamber.

Microemulsions can be used for synthesis of metallic, semiconductor, silica, barium sulfate, magnetic, and superconductor nanoparticles. By controlling the very low interfacial tension (˜10⁻³ mN/m) through the addition of a cosurfactant (e.g., an alcohol of intermediate chain length), these microemulsions are produced spontaneously without the need for significant mechanical agitation. The technique is useful for large-scale production of nanoparticles using relatively simple and inexpensive hardware. High energy ball milling has been used for the generation of magnetic, catalytic, and structural nanoparticles.

It is often important to achieve the controlled generation of monodispersed nanoparticles with size variance so small that size selection by centrifugal precipitation or mobility classification is not necessary. Among all the synthesis techniques discussed above, gas-phase synthesis is one of the best techniques with respect to size monodispersity, typically achieved by using a combination of rigorous control of nucleation-condensation growth and avoidance of coagulation by diffusion and turbulence as well as by the effective collection of nanoparticles and their handling afterwards. The stability of the collected nanoparticle powders against agglomeration, sintering, and compositional changes can be ensured by collecting the nanoparticles in liquid suspension. Surfactant molecules have been used to stabilize the liquid suspension of metallic nanoparticles. Alternatively, inert silica encapsulation of nanoparticles by gas-phase reaction and by oxidation in colloidal solution has been shown to be effective for metallic nanoparticles.

Approaches have been developed for the generation of monodisperse nanoparticles that do not require the use of a size classification procedure. Monodispersed gold colloidal nanoparticles with diameters of about 1 nm can be prepared by reduction of metallic salt with UV irradiation in the presence of dendrimers. Poly(amidoamine) dendrimers with surface amino groups of higher generations have spherical 3-D structures, which may have an effective protective action for the formation of gold nanoparticles.

One production method that is suitable for the production of these materials is the Tesima® process (described in WO 01/78471 and WO 01/58625) where a high temperature DC plasma is used to generate plasma within an inert gas envelope. Materials (either pre-produced feedstock or mixed feedstock), or liquids, can be placed into the plasma causing them to vaporise very rapidly. The resultant vapour then exits the plasma where it is then cooled by quantities of cold gas. These gases can be either inert (such as argon or helium) or can be air, or can have trace components to develop the chemistry/morphology/size that is required. The rapid cooling (greater than 100,000 degree a second) then freezes the particle for subsequent cooling and collection using a combination of techniques that can include solid or fabric filters, cyclones and liquid systems. The materials can also be collected directly into containers under either inert gas or into various liquids.

In one embodiment of the invention, the nanoparticles are prepared by a process which comprises the generation of plasma within an inert gas envelope and the insertion into the plasma of a substance and/or liquid comprising an element or elements or compounds of said element or elements, or a mixture thereof, followed by the gas cooling of the resultant vapour upon exit from the plasma.

The reduction and/or prevention of virus transmission may be defined as a reduction on viral titre of at least 90% following administration of a composition of nanoparticles as defined herein to a preparation of virus. Preferably the reduction on viral titre is at least 93%, 94% or 95%, most preferably 98%, 99% or 100%. Reduction and/or prevention of virus transmission is demonstrated by the inactivation of virus upon contact with the nanoparticles.

A reduction in viral titre of 70% or less is not an effective reduction sufficient to avoid infection. The present invention provides a means for reducing viral titre such that infection is prevented or avoid to a significant extent.

Viral titre is a quantitation of the number of virus particles in a given sample. It may be performed by using the Hemagglutination Assay (HA). Viral families have surface or envelope proteins that are able to agglutinate animal Red Blood Cells (RBC) and bind to N-acetylneuraminic acid residues on the cell surface of the RBCs. The RBC will form a type of lattice following viral binding which can be quantitated.

The HA procedure is an easy, simple and rapid method and can be applied to large amounts of samples. The detailed conditions depend on the type of virus. Some viruses bind RBCs only at certain pH values, others at certain ionic strengths. However, these are well known to the person skilled in the art and can be readily identified according to the virus in question. A virus dilution will be applied to a RBC dilution for a suitable period of time under appropriate conditions. Subsequently, the formation of lattices will be counted and the titre calculated.

The present invention provides a means to reduce the viral titre of a virus, preferably the virus is selected from the group consisting of Influenza, Measles, Coronavirus, Mumps, Marburg, Ebola, Rubella, Rhinovirus, Poliovirus, Hepatitis A, Smallpox, Chicken-pox, Severe Acute Respiratory Syndrome virus or SARS virus (also referred to as SARS coronavirus), Human Immunodeficiency Virus (HIV) and associated non-human animal immunodeficiency retroviruses such as Simian Immunodeficiency Virus (SIV), Rotavirus, Norwalk virus and Adenovirus. Norwalk virus includes its surrogate Feline Calicivirus. Influenza viruses include both human and avian forms of the virus.

The present invention therefore also provides a composition comprising nanoparticles as described above for use as an antiviral agent. The nanoparticles may suitably be formulated in an appropriate carrier, coating or solvent such as water, methanol, ethanol, acetone, water soluble polymer adhesives, such as polyvinyl acetate (PVA), epoxy resin, polyesters etc, as well as coupling agents, antistatic agents. Solutions of biological materials may also be used such as phosphate buffered saline (PBS), or simulated biological fluid (SBF).

The concentration of the nanoparticles in the solution may in the range of from 0.001% (wt) to about 20% (wt).

In one embodiment of this aspect of the invention there is provided the use of nanoparticles of average particle size up to 100 nm of a compound of general formula MₙX_{y}, where M is
(i) a metal selected from the group consisting of Calcium (Ca), Aluminium (Al), Zinc (Zn), or Copper (Cu);
or (ii) a non-metal selected from the group consisting of Silicon (Si), or Carbon (C);
   in which n is equal to 1, or 2,

### and X is

(iii) a non-metal selected from the group consisting of Oxygen (O), Nitrogen (N), or Carbon (C);
or (iv) an anion selected from the group consisting of phosphate (PO₄³⁻), hydrogen phosphate (HPO₄²⁻), dihydrogen phosphate (H₂PO₄⁻), carbonate (CO₃⁻), silicate (SiO₄²⁻), sulphate (SO₄²⁻), nitrate (NO₃⁻), nitrite (NO₂⁻);
   in which y is equal to 0, 1, 2 or 3;
   for reducing and/or preventing virus transmission.

Reduction and/or prevention of virus transmission includes the prevention of viral infection of a subject with a virus, in addition to the prevention of viral transmission from a first location to a second location, for example from an external space to an internal lumen, or the prevention of viral transmission through a barrier material. The subject may be a human or a non-human animal, suitably a non-human mammal. The present invention may therefore find application in the fields of human medicine and animal veterinary medicine as well as in the field of infection control in a non-medical context, such as a prophylactic against viral transmission.

According to a second aspect of the invention, there is provided a method for the reduction and/or prevention of virus transmission, comprising applying a composition of nanoparticles as defined above to an article of protective clothing.

The nanoparticles used in accordance with this aspect of the invention may be formulated in a composition as described above.

The coating process may be by any generally suitable means, such as for example, spray coating, electro-spray coating, dipping, plasma coating.

Such articles of protective clothing may be prepared from any suitable fibre or fabric, such as natural or artificial fibres. Natural fibres include cotton, wool, cellulose (including paper materials), silk, hair, jute, hemp, sisal, flex, wood, bamboo. Artificial fibres include polyester, rayon, nylon, Kevlar®, lyocell (Tencell®), polyethylene, polypropylene, polyimide, polymethyl methacrylate, Poly (Carboxylato Phenoxy) Phosphazene PCPP, fibre glass (glass), ceramic, metal, carbon. The article of clothing may be selected from the group consisting of face masks (surgical masks, respirator masks), hats, hoods, trousers, shirts, gloves, skirts, boilersuits, surgical gowns (scrubs) etc. Such clothing may find particular use in a hospital where control of infection is important.

According to a third aspect of the invention, there is provided a method for the reduction and/or prevention of virus transmission, comprising applying a composition of nanoparticles as defined above to a filter. The application of the composition of nanoparticles may be as described in relation to the second aspect of the invention.

The filter may be prepared from any suitable natural or artificial material as described above in relation to the second aspect of the invention.

The filter may be an air filter. An air filter is a device which removes contaminants, often solid particles from air. Air filters are often used in diving air compressors, ventilation systems and any other situation in which air quality is important, such as in air-conditioning units. An air filter includes devices which filter air in an enclosed space such as a building or a room, as well as apparatus or chambers for handling viral materials. Other articles which perform a protective function such as curtains or screens may therefore also be considered as air filters. An air filter according to this aspect of the invention may therefore also be prepared according to the second aspect of the invention.

Air filters may be composed of paper, foam, cotton filters, or spun fibreglass filter elements. Alternatively, the air filter may use fibers or elements with a static electric charge. There are four main types of mechanical air filters: paper, foam, synthetics and cotton.

An example of pleated-paper air filters designed for in-duct use with home heating, ventilation and air-conditioning (HVAC) systems is the 3M “Filtrete” product.

Polyester fiber can be used to make web formations used for air filtration. Polyester can be blended with cotton or other fibers to produce a wide range of performance characteristics. In some cases Polypropylene may be used. Tiny synthetic fibers knows as micro-fibers may be used in many types of HEPA (High Efficiency Particulate Air Filter) filters. High performance air filters may use oiled layers of cotton gauze.

Alternatively, the filter may be used to filter liquids. Such filters may be composed of any suitable fibre as described above. Filters used to filter liquids may be used to filter potable liquids for human or animal consumption, water for general domestic use, fluids for medical use, such as plasma or saline solutions, or pharmaceutical formulations for injection, or other biological liquids which may come into contact with a patient.

According to a fourth aspect of the invention, there is provided an article of protective clothing composed of fibres in which said fibres are coated with a composition of nanoparticles as defined above. The article of protective clothing may suitably be a face mask. Such masks may cover the whole face of the user or a part thereof, suitably the external areas of the nose and/or mouth of the wearer.

According to a fifth aspect of the invention, there is provided a filter composed of fibres in which said fibres are coated with a composition of nanoparticles as defined above. Suitably the filter may be an air filter.

In aspects of the invention relating to articles of protective clothing or filters, it should be noted that the articles of clothing or filters may be made of mixed fibres from any source as described above.

In a preferred embodiment of the present invention there is provided a face mask or a filter composed of a fibrous material which has been coated with a nanoparticle composition as defined herein.

The present invention also provides the use of mixed nanoparticles of zinc oxide (ZnO) and titanium dioxide (TiO₂) for reducing and/or preventing virus transmission. Such mixed nanoparticles of the invention may also be used in methods as described above, or in filters as described above, or articles of protective clothing as described above.

Preferred features for the second and subsequent aspects of the invention are as for the first aspect mutatis mutandis.

The present invention will now be further described with reference to the following Examples and Drawings which are provided for the purposes of illustration only and are not to be construed as being limiting on the invention. Reference in the Examples is made to a number of Drawings in which:

FIG. 1 shows Nano scaled electron microscope images of Flu and SARS viruses

FIG. 2 shows Masks to prevent air borne particles and virus. The gaps between fabrics are more than 10 μm in modern fabric masks.

FIG. 3 shows SEM image of the filtration fabrics used for public building such as university research buildings, shopping centres and hospitals, etc.

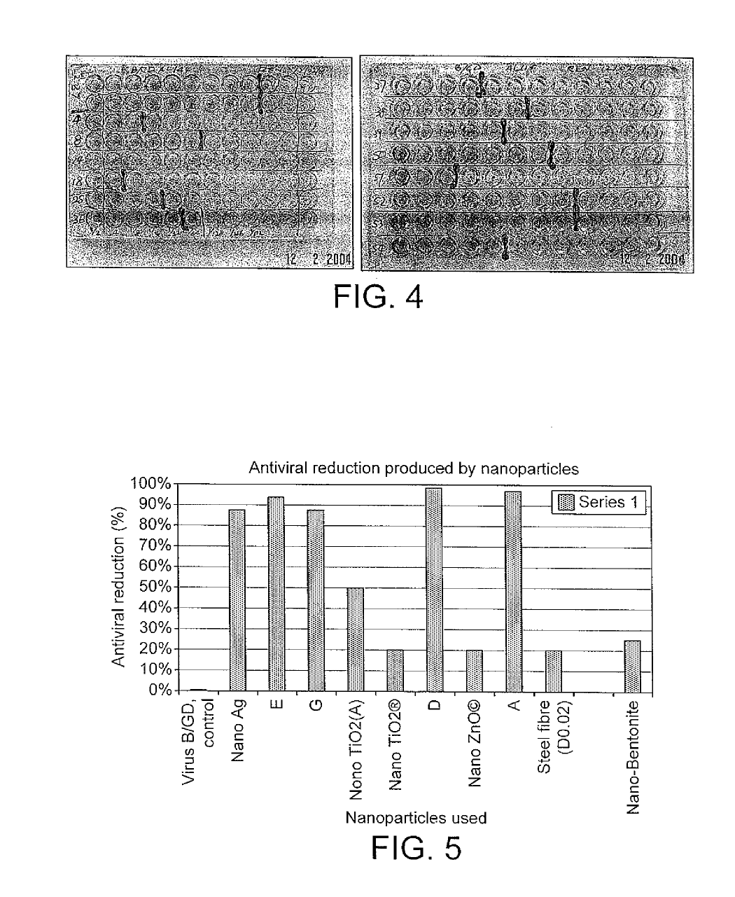

FIG. 4 shows Two example plates of HA assay tests of using nanomaterials as antiviral agents.

FIG. 5 Test results showing antiviral effects of viral reductions caused by the nanoparticles of different metals, metal oxides and compounds (the control is on the left).

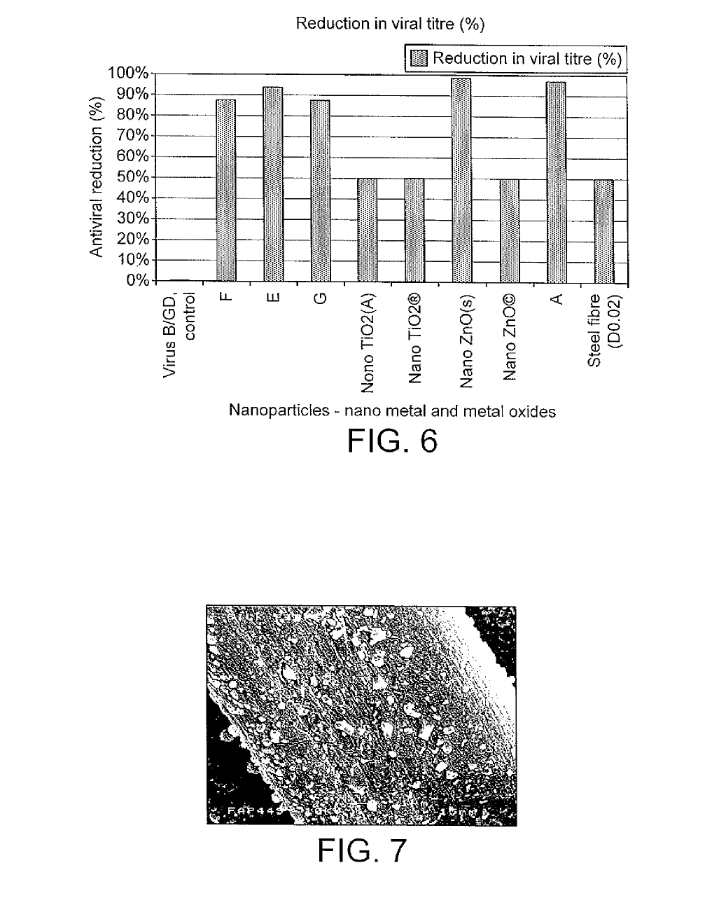

FIG. 6 Viral reductions of different metals and metal oxides tested, including nanoparticles of, nano Silver, nano TiO₂,nano ZnO, nano Cu, nano Ni, nano TiO₂ (both Anitase and Rotal crystals), nano ZnO, nano SiO₂ and Steel, etc.

FIG. 7 shows coated single fibre with a mixture of nano and micro scaled particles/compounds for antimicrobial trials.

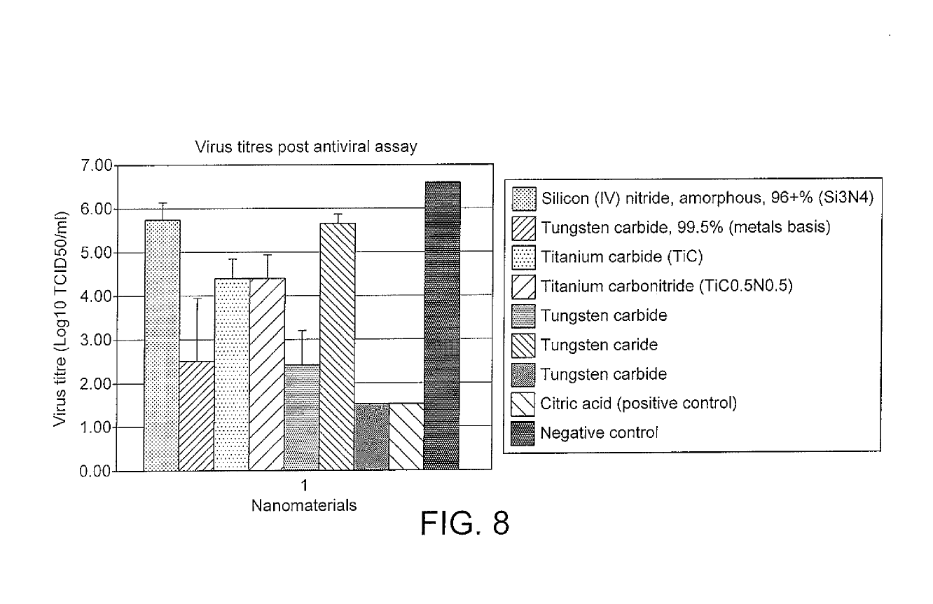

FIG. 8 shows percentage reduction in virus titre for nanocompounds of silicon (IV) nitride, tungsten carbide, titanium carbide, titanium carbonitride. Amount of avian H5N1 Influenza NIBRG-14 virus quantified after reacting with the test nanomaterials in the virucidal assay (Log₁₀ TCID₅₀/ml).

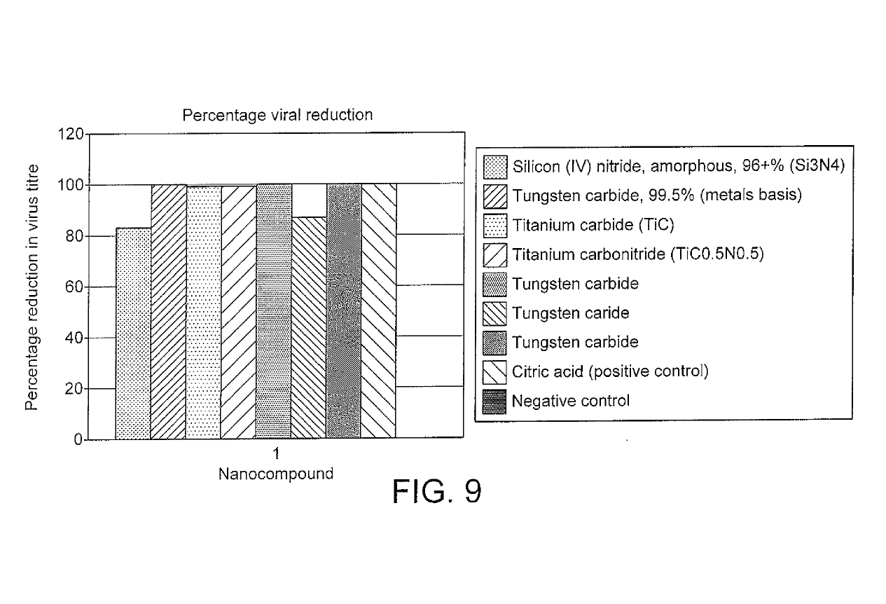

FIG. 9 shows virus titres post anti-viral assay of virus titre (Log₁₀ TCID50/ml) for nanocompounds of silicon (IV) nitride, tungsten carbide, titanium carbide, titanium carbonitride. Percentage reduction in avian H5N1 Influenza NIBRG-14 virus after reacting with the test nanomaterials in the virucidal assay (%).

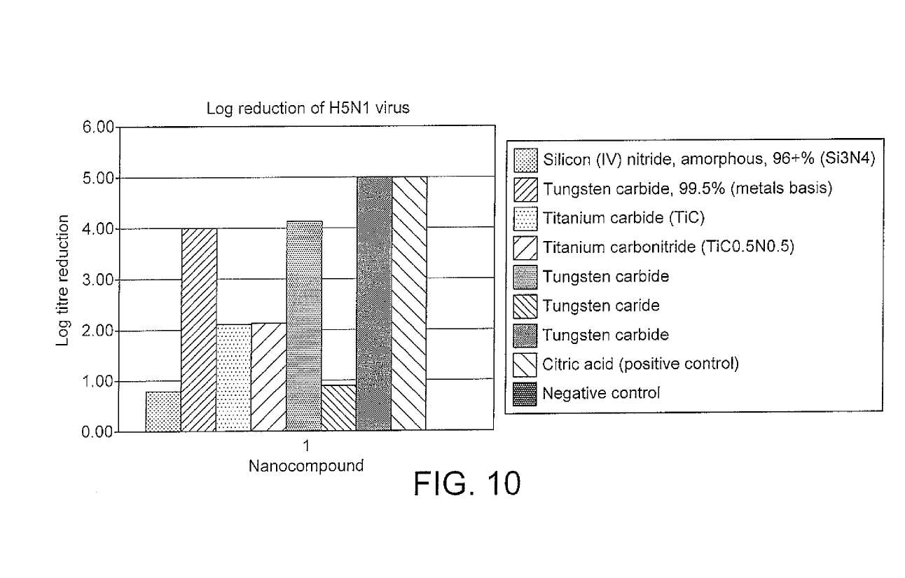

FIG. 10 shows log reduction of H5N1 virus (results expressed as Log titre reduction with respect to nanocompound) for nanocompounds of silicon (IV) nitride, tungsten carbide, titanium carbide, titanium carbonitride. Viral titre reduction in avian H5N1 Influenza NIBRG-14 virus after reacting with the test nanomaterials in the virucidal assay (Log₁₀, TCID₅₀/ml).

### EXAMPLE 1

### Preliminary Studies on Anti-Viral Properties of Nanomaterials

More than 60 different materials were screened through biological evaluation using the HA procedure in order to quantify the amount of influenza virus, haemagglutanin (HA) antigen present in a sample.

### Materials:

96 U well or V bottom micro titre plate
Turkey red blood cells (TRBC's)
Phosphate Buffered Saline (PBS)
50 ml pipette
Disposable pipette tips

### Method

1. Add 50 ml PBS to all the walls from row 2-12 of the micro titre plate
2. Add PBS to the first wall, the amount dependant on the diluents of the sample required.
3. The virus sample treatment: adding water solution or suspension containing ˜0.1 to 1% nanoparticles or testing materials.
4. Add the sample in an appropriate volume to the wells in the first raw. (Each sample and dilution range should be done in duplicate).
5. Dilute samples across the plate from rows 1-11.
6. Make up a 0.5% TRBC's solution in PBS.
7. Add 50 ml of 0.5% TRBC's to all the wells being used and row 12 (RBC control).
8. Place the plates on a mixer platform for 30 seconds to facilitate even distribution of TRBC's.
9. Leave the plates for 30 minutes at room temperature to settle.
10. Read the plates.
11. Plate should be read and graded as follows:

### Negative Result

A pellet should be formed on the bottom of the wells. When the plate is tilted to 45°, the pellet should form a streak as the TRBC's move slowly downwards. This shows there is not a sufficient amount of virus virons to crosslink the TRBC's.

### Positive Result

A positive result is seen when the TRBC's agglutinated and form a diffuse matrix through the well. This shows that virus virons are present in sufficient amounts to cross-link the TRBC's (two test plate as shown in FIG. 4).

Collapsed haemagglutination may occur when the titre of the virus is comparatively high in relation to TRBC's. This can appear on pellet in the bottom of a well, but on tilting plate it will remain in place. Should this occur, it is advisable to repeat the assay using a lower titre.

The end point of the assay is defined as the lowest contraction of the virus (highest diluents) that still causes Haemagglutination.

The titre of the virus is recorded as Haemagglutination unites (HAV) and is directly related to the dilution in the end point of well.

### EXAMPLE 2

### Haemagglutination Assays Using Different Natural and Man Made Materials

To test this, the screening of virus reactions to natural and manmade nanomaterials was initiated using HA assay. The purpose was to identify and classify the most effective nanomaterials for inactivating viruses to protect against Flu and SARS. Some natural and manmade nanomaterials were identified possessing special properties to disable or inactivate the standard NB type flu viruses.

In the tests, neat virus B/GD AL444, VCI/256 and other flu virus were used to test against different materials.

Neat HA (NHA): 1/512 at room temperature, 1/256-1/512 at 37° C.; the virus reaction to materials are shown as reduction in virus titre % (virus-material titration-VTMHA). During the test, after the virus solution was mixed with materials, the mixture was left in the room temperature 20° C. or in an incubator for 30 minutes at 37° C. More than 20 elemental nanoparticles and their compounds have been tested to date, some of them getting over 90% virucidal rates. 12 out of over 60 samples were HA tested are shown in Table 1.

One of the results of the preliminary B/GD virus reaction tests was a reduction in virus quantity in the HA assay by adding small percentages of nanomaterial. These results indicate a reduction in viral activity. Some of the nanomaterials could have completely disabled/inactivated the viral capability of binding to red blood cells.

FIG. 5 and FIG. 6 show the test results of virus level changes (%) by adding nanoparticles of different metal, metal oxide and their compounds in relevant to Table 1 and Table 2.

TABLE 1
HA test results using nanomaterials as antiviral agents

| | Reduction in viral titre (%)* | | | | |
|---|---|---|---|---|---|
| | | | | | Flu viruses |
| | 21° C., | | | | VC1/256/, |
| | B/GD | 37° C., 30 min. | Flu virus 1 | | A/Panama/2007/99 |
| Nanomaterials tested | AL444 | B/GD AL444-1 | (VC1/256) | Flu virus 2 | Al694 |
| A Type | 96.8-98.4% | 96.8-98.4% | 96.8-98.4% | 96.8-98.4% | 98.4% |
| B Type | 96.8-100% | 96.8-100% | | | 98.4% |
| C Type | 93.8% | 93.8% | 96.9% | 93.8% | 96.9% |
| ZnO - micron scaled | 20% | 0.0% | ~ | ~ | ~ |
| D Type | 99.2% | 93.8% | | 100% | 99.6% |
| E Type | 96.7% | | | 75-96.8% | 96.8% |
| Nano Ag (F) | 80% | 80% | | 87.3% | 87.25-93.8% |
| Nano bentonites (H) | 50% | 50% | 0% | 0% | ~ |
| Nano TiO₂ (a/r) (I) | 50% | 50% | ~ | 0% | ~ |

*If the reduction in viral titre produced by the nanomaterial is less than 70%, the nanomaterial was considered to have no antiviral effect.
A Type: nano Al, nano Al₂O₃ and related compounds.
B Type: nano Si, nano SiO₂ and related compounds.
C Type: nano SiC and related compounds.
D Type: nano Zn, ZnO and related compounds.
E Type: nano Cu, CuO, Cu₂O and related compounds.
F Type: nano Ag and it compounds.
G Type: nano Ni, and NiO₂ and related compounds.
H: nano Bentonite particles.
I: nano TiO₂ related materials.

TABLE 2
HA test results using nanomaterials as antiviral agents (antiviral nano composites)

| | Reduction in viral titre (%) | | | | |
|---|---|---|---|---|---|
| | | | | | Flu viruses |
| | | | | | VC1/256/, |
| | 21° C., | 37° C., 30 min. | | | A/Panama/ |
| | B/GD | B/GD | Flu virus 1 | | 2007/99 |
| Material tested | AL444 | AL444-1 | (VC1/256) | Flu virus 2 | Al694 |
| Nano Al- | 96.8-98.4% | 96.8-98.4% | 96.8-98.4% | 96.8-98.4% | 98.4% |
| oxide/related | | | | | |
| compounds | | | | | |
| Nano Si and | 96.8-100% | 96.8-100% | | | 98.4% |
| related | | | | | |
| compounds | | | | | |
| Nano Si—C | 93.8% | 93.8% | 96.9% | 93.8% | 96.9% |
| compounds | | | | | |
| Ca—P related | 98.4% | 99.2% | 93.8% | 99.6% | 99.6-100% |
| compounds | | | | | |
| Ca—C compounds | 93.8% | 96.9% | 93.4% | | |
| Nano Zn related | 99.22% | 93.8% | | 100% | 99.6% |
| compounds | | | | | |
| Nano Cu related | 96.7% | | | 75-96.8% | 96.8% |
| compounds | | | | | |
| Nano Ag | 80% | 80% | | 87.3% | 87.25-93.8% |
| Nano Ni related | | | | 87.3% | 87.3% |
| compounds | | | | | |
| Nano Bentonite | 50% | 50% | | | |
| NanoTiO2 related | 50% | 50% | | | |
| Micro SiO₂ | | | | 75% | 75-87.3% |

Analysis of HA Assay Results

In order to find novel materials for facial masks and filters which can interact with Flu/SARS viruses, screening of virus reactions to nano-materials using biological assays was undertaken. Nanomaterials have been identified possessing special properties to disable or inactivate the standard flu viruses. The short term tests have been concentrated on screening potential nanomaterials and classifying most effective materials to be used for inactivating viruses in the applications of facial masks and filters.

Without being bound by theory, the current assumption is that small sized and highly activated nanoparticles (such as SiO₂) which may be either hydrophilic or hydrophobic (or both at the same time), nano TiO₂ particles, metal particles (Au, Cu) and ceramic particles (SiC, Al₂O₃) that are the same size as viruses may be taken up by viruses.

The strong surface functionality of the nanomaterials may mimic the interaction of animal cells with viruses

The present study has explored the use of nanoparticles for use as virucidal agents and has identified the most effective ones as targets for developing coating materials specifically to absorb viruses. A low cost facial mask coated with antiviral nanoparticles may stop the viruses by attraction or contaction but significantly will then provide for inactivation of the virus. It is observed that the nanomaterials smaller than 100 nm are more effective in antiviral activities. A single fibre coated with antiviral nanoparticles under SEM observation is shown in FIG. 7. The particles are a mixture of different nanomaterials and compounds. Nanomaterials according to the present invention can be applied to enclosed ventilation fabrics for public buildings, hospitals, and modes of transport such as vehicles, cars, trains, ships and aeroplanes. The nanoparticles will also find use in medical applications, such as in filtering materials, i.e. in filtration of biological fluids such as plasma, blood, milk, semen etc to inactivate virus.

The antiviral nanoparticles may be coated on fabrics and surfaces of different products such as furniture, paints/coatings, book covers, computer keyboard in order to produce products with anti-viral properties. Such products will provide a low cost viral-free environment for hospitals, children, patients and the elderly. Further uses may include air ventilation systems for enclosed environments such as passenger aeroplanes, large buses and cars for preventing the entry or outlet of nanoparticles and airborne influenza viruses and other infectious viruses.

One of the preliminary B/GD virus reactions to nanomaterials shows the reductions of virus quantity in the HA assay by adding small percentages of nanomaterials which shows how the nanomaterials may be used to prevent viral transmission. Some of the materials completely disable or inactivate virus capability of binding to red blood cells in the HA assay.

The preliminary results of the B/GD virus screen proved a quantity reduction of virus in the HA assay by adding small percentages (<1%) of nanomaterials or compounds.

The test results of virus level changes (by percentages) adding different nanoparticles such as inorganic nano compounds which may be coated with metals and metal oxides such as calcium phosphates, and ceramics such as SiC, alumina, metals and metal oxides as nano Ag, Cu, Zn, Al, nano-scaled CuO, Cu₂O, Al₂O₃, TiO₂, nano ZnO, etc. The complex nano clusters of combinations of inorganic and mineral compound coated with metals and metal oxides are also part of the present invention, such as nano compounds containing mixed element groups of C—P—Ag—Zn, C—P—Cu—S, C—P—Cu—Ni—S, C—Si—Ag—Zn, C—Si—Cu—S, C—Si—Cu—Ni, etc as shown in Table 3 and Table 4.

The current results has identified a set of nanomaterials with improved anti-viral activity over other nanomaterials such as silver. The present research also shows the benefit of the use of multiple materials of each nanomaterial, producing nano-scaled clusters (e.g. such as nanoparticle materials available from manufacturers such as QinetiQ Nanomaterials Limited), nanoparticle combinations of inorganic/organics, mineral compounds and coated with metals and metal oxides.

The present results show that 20 nanoparticles have been tested plus many types of compound materials: nano Ag (poor), TiO₂ (poor), ZnO (good), Alumina (good), and Al-related compounds such as Al-phosphates, Cu and Cu related oxides and compounds, Ca²⁺ related compounds such as Ca-phosphates, Ca-silicates and Ca-carbonates, Si related compounds such as SiO₂ and SiC, and P related compounds such as Al-phosphates and active carbons all demonstrating over 90% virucidal rates.

Compounds and mixtures of multi-elements multi-oxides according to the present invention may be used to deal with transmission of multiple viruses and potential viral contamination, for example, using clusters of compounds to deal with different flu viruses and SARS viruses.

TABLE 3
Materials for use in antiviral applications.

| | | Chemically or |
|---|---|---|
| Metal and non-metal | Metals and metal | physically combined |
| elements | oxides | nanoparticle mixtures |
| B, C | TiO₂, Cu₂O, CuO, | C—P—Ag—Zn |
| Al, Si, P | ZnO,, NiO, Al₂O₃, | C—P—Cu—S |
| Ca, Ti, Cr, Mn, Fe, Co | FeₓO_{y}, CoₓO_{y}, | C—P—Ci—Ni—S |
| Ni, Cu, Zn | SiₓO_{y} | C—Si—Ag—Zn |
| Ag, W, Cd | | C—Si—Cu—S |
| | | C—Si—Cu—Ni |
| | | C—W—Cu—Ag—Zn |
| | | W—Ti—B—C |

TABLE 4
Combinations of materials for antiviral applications.

| Metal and non-metal | Metals and non | |
|---|---|---|
| elements | metal oxides | |
| coatings or clusters | coatings or clusters | Compound materials |
| B, C | TiO₂, Cu₂O, CuO, | CaPO₄ + |
| Al, Si, P | ZnO,, NiO, Al₂O₃, | CaCO₃ + |
| Ca, Ti, Cr, Mn, Fe, Co Ni, | FeₓO_{y}, CoₓO_{y}, | SiC + |
| Cu, Zn | SiₓO_{y} | CaSiO₄ + |
| Ag, W, Cd | | SiO₂ + |
| | | Al-phosphate |
| | | WC |
| | | TiN |
| | | Bn |
| | | TiBN |

### EXAMPLE 3

### Preliminary Studies on Virucidal Efficacy of Nanoparticles on Avian H5N1 Influenza NIBRG-14 Virus

This example shows the results of studies to test the virucidal efficacy of test nanomaterials against avian H5N1 Influenza NIBRG-14 virus using MDCK cells. In the tests, avian H5N1 Influenza NIBRG-14 virus was used to test against different materials.

The amount of virus tested against in the “reaction mixture” was 10^{6.5}TCID₅₀/ml (Tissue Culture Infective Units). The affect of the nanomaterials on the virus are shown as reduction in virus titre (% and Log₁₀ TCID₅₀/ml). Virus was diluted in distilled water (1:10 dilution from a stock of 10^{7.5}TCID₅₀/ml virus grown in eggs). Virus (200 μl) was then added to the nanomaterials to form the “reaction mixture”. The reaction mixture (nanomaterial and virus solution) was lightly vortexed (mixed for 5 seconds) at room temperature (20° C.), and then incubated for a further 30 minutes at room temperature while being shaken on a plate shaker to ensure continual contact of the nanomaterials with the virus particles.

Eight test nanomaterials were selected for analysis of virucidal action from the most promising materials previously tested in the HA assay. At the end of the 30 minute incubation the reaction mixtures were centrifuged to separate the nanomaterials from the virus and then added to cell maintenance media (at a 1:10 ratio) in preparation for infecting the MDCK cells. The virus was then quantified by making serial dilutions of the reaction mixture on MDCK cells to generate the “infective” titre (Log₁₀ TCID_{5c}/ml). A “negative control” (no nanomaterial was mixed with the virus) and a “positive control” of citric acid (a solution with a pH of approximately 3.5) were used to check for the performance of the assay.

### Results

These results show reductions in viral activity from the virucidal experiment. Some of the test nanomaterials exhibited good virucidal efficacy and reduced virus infectivity below the detectable limits of the assay. Comparing the amount of virus in the negative control (virus but no nanomaterial) with the reaction mixtures containing each of the nanomaterials gave the reduction in amount of infective virus (shown as Log₁₀ TCID₅₀/ml or %). The positive control (low pH) reduced the infectivity of the virus to below detectable limits of the assay such that no virus was observed to remain.

FIG. 8 shows the amount of infective virus present in the test reaction mixture at the end of the reaction time (Log₁₀ TCID₅₀/ml). FIGS. 9 and 10 shows the virucidal efficacy of the test nanomaterials as a percentage reduction in infective titre (%) and as a reduction in viral titre respectively (Log₁₀ TCID₅₀/ml). The results of the test reactions and the reductions in amount of infective virus (titre, %, and Log₁₀ TCID₅₀/ml) by adding the test nanomaterials are shown in Table 5 which reports the amount of avian H5N1 Influenza NIBRG-14 virus quantified after reacting with the test nanomaterials in the virucidal assay.

TABLE 5

| | | Mean Test | Mean Log | % Viral |
|---|---|---|---|---|
| Product Name | Batch | titre | reduction | reduction |
| Silicon (IV) | R31 | 6.50 | 0.78 | 83.400 |
| nitride, | | | | |
| amorphous, 96+% | | | | |
| (Si3N4) | | | | |
| Tungsten carbide, | R32 | 6.50 | 4.00 | 99.990 |
| 99.5% (metals | | | | |
| basis) | | | | |
| Titanium carbide | R33 | 6.50 | 2.11 | 99.223 |
| (TiC) | | | | |
| Titanium | R34 | 6.50 | 2.11 | 99.223 |
| carbonitride | | | | |
| (TiC0.5N0.5) | | | | |
| Tungsten Carbide | R35 | 6.50 | 4.11 | 99.992 |
| Tungsten Carbide | R36 | 6.50 | 0.89 | 87.120 |
| Tungsten Carbide | R37 | 6.50 | 5.00 | 99.999 |
| Citric Acid | n/a | 6.50 | 5.00 | 99.999 |
| (positive control) | | | | |
| Negative control | n/a | 6.50 | 0.00 | 0.00 |

The nanomaterials tested (results shown in Table 5) have size variations from 5 nm to 100 nm. Tungsten carbide (R32) has a purity of 99.5%. Tungsten carbides (R36, R37 and R38) were manufactured with different plasma conditions and cooling speed/steps and particle size distributions.

What is claimed is:

1. The use of nanoparticles of a compound of general formula MₙX_{y}, where M is
(i) a metal selected from the group consisting of Calcium (Ca), Aluminium (Al), Zinc (Zn), Nickel (Ni), Tungsten (W), or Copper (Cu);
or (ii) a non-metal selected from the group consisting of Silicon (Si), Boron (B) or Carbon (C);
in which n is equal to 1, 2, or 3,
and X is
(iii) a non-metal selected from the group consisting of Oxygen (O), Nitrogen (N), or Carbon (C);
or (iv) an anion selected from the group consisting of phosphate (PO₄³⁻), hydrogen phosphate (HPO₄²⁻), dihydrogen phosphate (H₂PO₄⁻), carbonate (CO₃), silicate (SiO₄²⁻), sulphate (SO₄²⁻), nitrate (NO₃⁻), nitrite (NO₂⁻);
in which y is equal to 0, 1, 2, 3, or 4;
for reducing and/or preventing virus transmission.

2. A use as claimed in claim 1, in which the nanoparticles have an average particle size up to 100 nm.

3. A use as claimed in claim 2, in which the nanoparticles have an average particle size in a range of from about 1 nm to about 90 nm

4. The use as claimed in claim 1, in which the compounds of the general formula MₙX_{y} are oxides, carbonates, silicates, carbides, nitrides and/or phosphates.

5. The use as claimed in claim 4, in which the compounds of the general formula MₙX_{y} are selected from the group consisting of aluminium oxide (Al₂O₃), silicon dioxide, (SiO₂), zinc oxide (ZnO), aluminium phosphate (AlPO₄), aluminium hydrogen phosphate (Al₂(HPO₄)₃), aluminium dihydrogen phosphate (Al(H₂PO₄)₃), calcium oxide (CaO), calcium carbonate (CaCO₃), calcium silicate (CaSiO₄), calcium phosphate (Ca₃(PO₄)₂), calcium hydrogen phosphate (CaHPO₄), or calcium dihydrogen phosphate (Ca(H₂PO₄), silicon nitride (SiN), silicon carbide (SiC), boron nitride (BN), tungsten carbide (WC) and titanium carbonitride (TiC_{0.5}N_{0.5}).

6. The use as claimed in claim 1, in which the nanoparticles comprise a mixed composition of at least two compounds of general formula MₙX_{y} as defined in claim 1.

7. The use as claimed in claim 5, in which the mixed composition of nanoparticles is:
Copper (Cu), copper (II) oxide (CuO) and/or copper (I) oxide (Cu₂O).

8. The use as claimed in claim 1, in which the nanoparticles comprise a mixed composition of a compound of general formula MₙX_{y} as defined in claim 1 and one or more of
Aluminium (Al), Silicon (Si), Zinc (Zn), or Nickel (Ni), or combinations thereof.

9. The use as claimed in claim 8, in which the nanoparticles comprise:
(i) Aluminium (Al) and aluminium oxide (Al₂O₃),
(ii) Silicon (Si) and silicon dioxide (SiO₂)
(iii) Silicon (Si) and Silicon carbide (SiC)
(iv) Zinc (Zn) and zinc oxide (ZnO), or
(v) Nickel (Ni) and nickel (II) oxide (NiO)
or combinations thereof.

10. The use of claim 1, in which the nanoparticles further comprise titanium dioxide (TiO₂).

11. The use of claim 10, in which the nanoparticles further comprise zinc oxide (ZnO) and titanium dioxide (TiO₂).

12. The use of nanoparticles as claimed in claim 1, in which the nanoparticles further comprise elements selected from the group consisting of: Boron (B), Carbon (C), Aluminium, (Al), Silicon (Si), Phosphorous (P), Calcium (Ca), Titanium (Ti), Chromium (Cr), Manganese (Mn), Iron (Fe), Cobalt (Co), Silver (Ag), Zinc (Zn), Copper (Cu), Sulfur (S), Nickel (Ni), Gold (Au), Zirconium (Zr), Ytterbium (Yb), Zirconium (Zr) or an oxide thereof or combinations thereof.

13. A use as claimed in claim 12, in which the nanoparticles further comprise at least one of the following combinations of elements:
C—P—Ag—Zn, C—P—Cu—S, C—P—Cu—Ni—S, C—Si—Ag—Zn, C—Si—Cu—S, C—Si—Cu—Ni, C—Cu—Zn—W, C—Cu—Zn—Ag, C—Cu—Zn—W—Ag, C—W—Ti—B, C—W—Ti—N, C—Ti—N, Si—N, Ti—N, Al—N, B—N, Al—B.

14. A use as claimed in claim 1, in which the nanoparticles further comprise at least one of the following oxides:
TiO₂, Cu₂O, CuO, ZnO, NiO, Al₂O₃, FeO, Fe₂O₃, Fe₃O₄, CoO, Co₃O₄, or Si₂O₃, or a combination thereof.

15. The use of mixed nanoparticles of zinc oxide (ZnO) and titanium dioxide (TiO₂) for reducing and/or preventing virus transmission.

16. The use as claimed in claim 1, in which the virus is selected from the group consisting of Influenza, Measles, Coronavirus, Mumps, Marburg, Ebola, Rubella, Rhinovirus, Poliovirus, Hepatitis A, Smallpox, Chicken-pox, SARS, HIV, Rotavirus, Norwalk virus and Adenovirus.

17. The use as claimed in claim 1 wherein the reducing and/or preventing virus transmission is from a first location to a second location.

18. The use as claimed in claim 17, wherein the first location is a surface of an inanimate product.

19. The use as claimed in claim 18, wherein the product is selected from furniture, paints/coatings, book cover or computer keyboard.

20. A method for the reduction and/or prevention of virus transmission from a first product surface to a second surface comprising applying to a product surface a composition of nanoparticles of a compound of general formula MₙX_{y}, where M is
(i) a metal selected from the group consisting of Calcium (Ca), Aluminium (Al), Zinc (Zn), Nickel (Ni), Tungsten (W) or Copper (Cu);
or (ii) a non-metal selected from the group consisting of Silicon (Si), Boron (B) or Carbon (C);
in which n is equal to 1, 2, or 3,
and X is
(iii) a non-metal selected from the group consisting of Oxygen (O), Nitrogen (N), or Carbon (C);
or (iv) an anion selected from the group consisting of phosphate (PO₄³⁻), hydrogen phosphate (HPO₄²⁻), dihydrogen phosphate (H₂PO₄₋), carbonate (CO₃), silicate (SiO₄²⁻), sulphate (SO₄²⁻), nitrate (NO₃⁻), nitrite (NO₂⁻);
in which y is equal to 0, 1, 2, 3, or 4.

21. An inanimate product coated with a composition of nanoparticles of a compound of general formula MₙX_{y},
where M is
(i) a metal selected from the group consisting of Calcium (Ca), Aluminium (Al), Zinc (Zn), Nickel (Ni), Tungsten (W) or Copper (Cu);
or (ii) a non-metal selected from the group consisting of Silicon (Si), Boron (B) or Carbon (C);
in which n is equal to 1, 2, or 3,
and X is
(iii) a non-metal selected from the group consisting of Oxygen (O), Nitrogen (N), or Carbon (C);
or (iv) an anion selected from the group consisting of phosphate (PO₄³⁻), hydrogen phosphate (HPO₄²⁻), dihydrogen phosphate (H₂PO₄₋), carbonate (CO₃), silicate (SiO₄²⁻), sulphate (SO₄²⁻), nitrate (NO₃⁻), nitrite (NO₂⁻);
in which y is equal to 0, 1, 2, 3, or 4.

22. An inanimate produce as claimed in claim 21, wherein the product is an item of furniture, paint/coating, a book cover or a computer keyboard.

23. A method for the reduction and/or prevention of virus transmission, comprising applying to an article of protective clothing a composition of nanoparticles of a compound of general formula MₙX_{y}, where M is
(i) a metal selected from the group consisting of Calcium (Ca), Aluminium (Al), Zinc (Zn), Nickel (Ni), Tungsten (W) or Copper (Cu);
or (ii) a non-metal selected from the group consisting of Silicon (Si), Boron (B) or Carbon (C);
in which n is equal to 1, 2, or 3,
and X is
(iii) a non-metal selected from the group consisting of Oxygen (O), Nitrogen (N), or Carbon (C);
or (iv) an anion selected from the group consisting of phosphate (PO₄³⁻), hydrogen phosphate (HPO₄²⁻), dihydrogen phosphate (H₂PO₄⁻), carbonate (CO₃), silicate (SiO₄²⁻), sulphate (SO₄²⁻), nitrate (NO₃⁻), nitrite (NO₂⁻);
in which y is equal to 0, 1, 2, 3, or 4.

24. A method for the reduction and/or prevention of virus transmission, comprising applying to a filter a composition of nanoparticles of a compound of general formula MₙX_{y}, where M is
(i) a metal selected from the group consisting of Calcium (Ca), Aluminium (Al), Zinc (Zn), Nickel (Ni), Tungsten (W) or Copper (Cu);
or (ii) a non-metal selected from the group consisting of Silicon (Si), Boron (B) or Carbon (C);
in which n is equal to 1, 2, or 3,
and X is
(iii) a non-metal selected from the group consisting of Oxygen (O), Nitrogen (N), or Carbon (C);
or (iv) an anion selected from the group consisting of phosphate (PO₄³⁻), hydrogen phosphate (HPO₄²⁻), dihydrogen phosphate (H₂PO₄⁻), carbonate (CO₃), silicate (SiO₄²⁻), sulphate (SO₄²⁻), nitrate (NO₃⁻), nitrite (NO₂⁻);
in which y is equal to 0, 1, 2, 3, or 4

25. An article of protective clothing composed of fibres in which said fibres are coated with a composition of nanoparticles of a compound of general formula MₙX_{y}, where M is
(i) a metal selected from the group consisting of Calcium (Ca), Aluminium (Al), Zinc (Zn), Nickel (Ni), Tungsten (W) or Copper (Cu);
or (ii) a non-metal selected from the group consisting of Silicon (Si), Boron (B) or Carbon (C);
in which n is equal to 1, 2, or 3,
and X is
(iii) a non-metal selected from the group consisting of Oxygen (O), Nitrogen (N), or Carbon (C);
or (iv) an anion selected from the group consisting of phosphate (PO₄³⁻), hydrogen phosphate (HPO₄²⁻), dihydrogen phosphate (H₂PO₄²⁻), carbonate (CO₃), silicate (SiO₄²⁻), sulphate (SO₄²⁻), nitrate (NO₃⁻), nitrite (NO₂⁻);
in which y is equal to 0, 1, 2, 3, or 4.

26. An article of protective clothing as claimed in claim 25, in which the article of clothing is composed of natural fibres, or the article of clothing is composed of artificial fibres.

27. An article of protective clothing as claimed in claim 25 or 26, in which said article of protective clothing is selected from the group consisting of a face mask, surgical mask, respirator mask, hat, hood, trousers, shirt, gloves, skirt, boiler-suit, and surgical gown.

28. A filter composed of fibres in which said fibres are coated with a composition of nanoparticles of a compound of general formula MₙX_{y}, where M is
(i) a metal selected from the group consisting of Calcium (Ca), Aluminium (Al), Zinc(Zn), Nickel (Ni), Tungsten (W) or Copper (Cu);
or (ii) a non-metal selected from the group consisting of Silicon (Si), Boron (B) or Carbon (C);
in which n is equal to 1, 2, or 3,
and X is
(iii) a non-metal selected from the group consisting of Oxygen (O), Nitrogen (N), or Carbon (C);
or (iv) an anion selected from the group consisting of phosphate (PO₄³⁻), hydrogen phosphate (HPO₄²⁻), dihydrogen phosphate (H₂PO₄⁻), carbonate (CO₃), silicate (SiO₄²⁻), sulphate (SO₄²⁻), nitrate (NO₃⁻), nitrite (NO₂⁻);
in which y is equal to 0, 1, 2, 3, or 4.

29. A filter as claimed in claim 28, in which the filter is composed of natural fibres, or artificial fibres.

30. A filter as claimed in any one of claim 28 or 29, in which the filter is an air filter.
